(19) United States
(12) Patent Application Publication
(10) Pub. No.: US 2004/0204481 A1
(43) Pub. Date: Oct. 14, 2004

(54) Activation of natural killer cells by adenosine A3 receptor agonists

(76) Inventor: Pnina Fishman, Herzliya (IL)

Correspondence Address: BROWDY AND NEIMARK, P.L.L.C., 624 NINTH STREET, NW, SUITE 300, WASHINGTON, DC, 20001-5303, US

(21) Appl. No.: 09/832,818

(22) Filed: Apr. 12, 2001

(51) Int. Cl. A61K 31/7076; A61K 31/695; A61K 31/52

(52) U.S. Cl. 514/460; 514/45; 514/63

(57) ABSTRACT

The present invention is based on the surprising finding that adenosine A3 receptor agonists (A3RAg) are capable of activating natural killer (NK) cells. In particular, methods and pharmaceutical compositions for activating NK cells in an individual comprising administering said individual with an effective amount of one or more A3RAg are provided herein.

A further method includes treatment of a disease comprising administering to an individual in need of such treatment NK cells a priori activated with an effective amount of at least one A3RAg. The NK cells may be either autologous cells or cells withdrawn from a donor individual.

Further provided are pharmaceutical compositions comprising one or more A3RAg in an amount effective to achieve a therapeutic effect, the therapeutic effect comprising activation of NK cells, the pharmaceutical composition optionally comprising physiologically acceptable additives.

### FIELD OF THE INVENTION

This invention relates to the therapeutic use of adenosine A3 receptor agonists for activating natural killer cells.

### BACKGROUND OF THE INVENTION

Natural killer cells (NK cells) were first identified in mice because of their capacity to rapidly lyse certain tumor cell targets. They are a small subset of peripheral blood lymphocytes, constituting 5 to 16 percent of the total lymphocyte population. These cells form a distinct group of lymphocytes with no immunological memory and are independent of the adaptive immune system.

NK cells mediate a variety of fractions that are important in human health and disease. For example, it has been found that NK cells are an important first line of defense against malignant cells and cells infected with viruses, bacteria, and protozoa. In addition, these cells participate in immunoregulation, haematopoiesis, reproduction and neuroendocrine interactions. The finding that NK cells effect the production of a number of cytokines, led to the suggestion that NK cells, like T cells, differentiate into discrete functional subsets with differing effects on adaptive immunity.

### SUMMARY OF THE INVENTION

It was found in accordance with the present that adenosine A3 receptor agonists (A3RAg) activate natural killer (NK) cells and that this activation was abolished in the presence of adenosine A3 receptor antagonists (A3RAn).

Adenosine is a ubiquitous nucleoside present in all body cells. It is released from metabolically active or stressed cells and subsequently acts as a regulatory molecule. It binds to cells through specific A1, A_{2A}, A_{2B} and A3 G-protein associated cell surface receptors, thus acting as a signal transduction molecule by regulating the levels of adenylyl cyclase and phospholipase C [Linden J. The FASEB J 5:2668-2676 (1991); Stiles G. L. Clin. Res. 38:10-18 (1990)].

In accordance with a first of its aspects, the present invention provides a method for activating NK cells in an individual, by providing said individual with an effective amount of one or more adenosine A3 receptor agonists (A3RAg).

The term “adenosine A3 receptor agonist” for purposes herein refers to any molecule capable of binding to the adenosine A3 receptor, thereby fully or partially activating said receptor. Some such molecules are provided hereinafter.

The “effective amount” (or “amount effective for”) for purposes herein is determined by such considerations as may be known in the art. The amount must be effective to achieve activation of NK cells at a detectable and preferably at a therapeutically effective level. A person versed in the art will know how to determine the effective amount depending, inter alia, on the type and severity of the disease to be treated and the treatment regime.

The term “activation of NK” for purposes herein refers to activation per se of the cytotoxic or cytostatic action of NK cells on foreign or abnormal cells or elevation of the cytotoxic or cytostatic action of pre-active (i.e. already active) NK cells on foreign or abnormal cells, as well as elevation of their other biological functions, such as stimulation of cytokine production

The present invention also provides a method for a therapeutic treatment comprising administering to an individual in need, one or more A3RAg in an amount effective for achieving a therapeutic effect, the therapeutic effect comprises activation of NK cells in said individual.

The term “treatment” for the purposes used herein refers to amelioration of undesired symptoms associated with a disease even without curing the disease, e.g. reduction of pain; prevention of the manifestation of such symptoms before they occur; slow down of the deterioration of symptoms or the progression of a disease; lessening of the severity or cure of the disease; acceleration of the natural or conventional healing processes; improvement of survival rate or more rapid recovery of a the individual suffering from a disease; prevention of a disease form occurring or a combination of two or more of the above.

Further, the invention provides a method for treatment of a disease comprising administering to an individual in need of such treatment NK cells a priori activated with an effective amount of A3RAg. Typically, such a method comprises withdrawing NK cells from the individual, exposing such cells to an effective amount of at least one A3RAg. Alternatively, the NK cells may also be from a donor individual. Such donated NK cells may be withdrawn after activation with the A3Rga in the donor individual or activated in vitro after withdrawal and before administering to the recipient individual.

The term “a priori activated” refers to activation of NK cells either in a cell or tissue culture or in an animal model wherein the cells, tissue or animal, respectively, are treated with an effective amount of A3RAg for activation of NK cells, and then cells or tissue preparations containing therein said activated NK cells are removed from the culture or from the animal for administration to an individual in need thereof. The activated NK cells administered to an individual are preferably, although not exclusively, autologous cells.

Yet further, the present invention provides a pharmaceutical composition comprising one or more A3RAg in an amount effective to achieve a therapeutic effect, the therapeutic effect comprising activation of NK cells.

### BRIEF DESCRIPTION OF THE DRAWINGS

In order to understand the invention and to see how it may be carried out in practice, a preferred embodiment will now be described, by way of non-limiting example only, with reference to the accompanying Figures, in which:

FIG. 1 shows a potentiation of the activity of human peripheral blood NK cells following incubation with 10 nM Cl-IB-MECA, while introduction of MRS-1220 A3 adenosine receptor antagonist, reversed the stimulatory effect of Cl-IB-MECA, indicating the specificity of the Cl-IB-MECA to the A3 receptor.

FIG. 2 shows that Cl-IB-MECA activates murine NK cells which is also time dependent, with a maximal activation after 4 days.

FIG. 3 shows increased NK cell activity in splenocytes derived from Cl-IB-MECA treated melanoma bearing mice.

FIG. 4 is an adoptive transfer experiment wherein melanoma bearing mice, engrafted with splenocytes derived from Cl-IB-MECA treated mice, exhibit decreased number of lung foci.

### DETAILED DESCRIPTION OF THE INVENTION

As will be shown in the following Examples, Cl-IB-MECA, an A3RAg, was found to activate NK cells, to a biologically significant level, both in vitro and in vivo. Evidently, this finding has a therapeutic value as NK cells participate in a number of biological processes, including defense against malignant and infectious diseases, immunoregulation, haematopoiesis, reproduction and neuroendocrine interactions.

In accordance with a first of its aspects, the present invention provides a method for activating natural killer (NK) cells in an individual, by administer to said individual with an effective amount of one or more A3RAg.

The characteristic and methods of preparation of some Adenosine A3 receptor agonists are described in detail in, inter alia, U.S. Pat. No. 5,688,774; U.S. Pat. No. 5,773,423, U.S. Pat. No. 5,573,772, U.S. Pat. No. 5,443,836, U.S. Pat. No. 6,048,865, WO 95/02604, WO 99/20284 and WO 99/06053, WO 97/27173, incorporated herein by reference.

According to one embodiment of the invention, the A3RAg is a compound of the general formula (I);

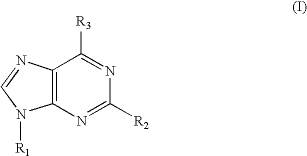

wherein,

R₁ represents an alkyl, hydroxyalkyl, carboxyalkyl or cyanoalkyl or a group of the following general formula (II):

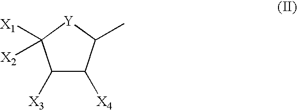

in which:

Y represents an oxygen, sulfur of carbon atom;

X₁ represents H, alkyl, R^{a}R^{b}NC(═O)— or HOR^{c}—, wherein p2 R^{a} and R^{b} may be the same or different and are selected from the group consisting of hydrogen, alkyl, amino, haloalkyl, aminoalkyl, BOC-aminoalkyl, and cycloalkyl or are joined together to form a heterocyclic ring containing two to five carbon atoms; and

R^{c} is selected from the group consisting of alkyl, amino, haloalkyl, aminoalkyl, BOC-aminoalkyl, and cycloalkyl;

X₂ is H, hydroxyl, alkylamino, alkylamido or hydroxyalkyl;

X₃ and X₄ represent independently hydrogen, hydroxyl, amino, amido, azido, halo, alkyl, alkoxy, carboxy, nitrilo, nitro, trifluoro, aryl, alkaryl, thio, thioester, thioether, —OCOPh, —OC(═S)OPh or both X₃ and X₄ are oxygens connected to >C═S to form a 5-membered ring, or X₂ and X₃ form the ring of formula (III):

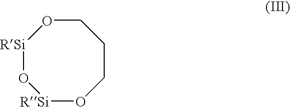

where R′ and R″ represent independently an alkyl group;

R₂ is selected from the group consisting of hydrogen, halo, alkylether, amino, hydrazido, alkylamino, alkoxy, thioalkoxy, pyridylthio, alkenyl; alkynyl, thio, and alkylthio; and

R₃ is a group of the formula —NR₄R₅ wherein

R₄ is a hydrogen atom or a group selected from alkyl, substituted alkyl or aryl-NH—C(Z)—, with Z being O, S, or NR² with R² having the above meanings; wherein when R₄ is hydrogen than

R₅ is selected from the group consisting of R- and S-1-phenylethyl, benzyl, phenylethyl or anilide groups unsubstituted or substituted in one or more positions with a substituent selected from the group consisting of alkyl, amino, halo, haloalkyl, nitro, hydroxyl, acetoamido, alkoxy, and sulfonic acid or a salt thereof; benzodioxanemethyl, fururyl, L-propylalanyl-aminobenzyl, β-alanylamino-benzyl, T-BOC-β-alanylaminobenzyl, phenylamino, carbamoyl, phenoxy or cycloalkyl; or R₅ is a group of the following formula:

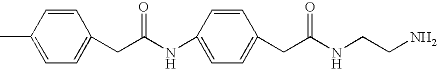

or when R₄ is an alkyl or aryl-NH—C(Z)—, then, R₅ is selected from the group consisting of heteroaryl-NR^{a}—C(Z)—, heteroaryl-C(Z)—, alkaryl-NR^{a}—C(Z)—, alkaryl-C(Z)—, aryl-NR—C(Z)— and aryl-C(Z)—; Z representing an oxygen, sulfor or amine;

or a physiologically acceptable salt of the above compound.

According to this embodiment, the A3RAg is more preferably a nucleoside derivative of the general formula (IV):

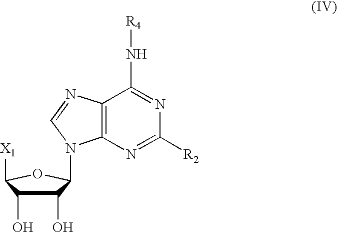

wherein X₁, R₂ and R₄ are as defined and physiologically acceptable salts of said compound.

The non-cyclic carbohydrate groups (e.g. alkyl, alkenyl, alkynyl, alkoxy, aralkyl, alkaryl, alkylamine, etc) forming part of the substituent of the compounds of the present invention are either branched or unbrancehd, preferably containing from one or two to twelve carbon atoms.

When referring to “physiologically acceptable salts” of the compounds employed by the present invention it is meant any non-toxic alkali metal, alkaline earth metal, and ammonium salts commonly used in the pharmaceutical industry, including the sodium, potassium, lithium, calcium, magnesium, barium ammonium and protamine zinc salts, which are prepared by methods known in the art. The term also includes non-toxic acid addition salts, which are generally prepared by reacting the compounds of this invention with a suitable organic or inorganic acid. The acid addition salts are those which retain the biological effectiveness and qualitative properties of the free bases and which are not toxic or otherwise undesirable. Examples include, inter alia, acids derived from mineral acids, hydrochloric, hydrobromic, sulfuric, nitric, phosphoric, metaphosphoric and the like. Organic acids include, inter alia, tartaric, acetic, propionic, citric, malic, malonic, lactic, fumaric, benzoic, cinnamic, mandelic, glycolic, gluconic, pyruvic, succinic salicylic and arylsulphonic, e.g. p-toluenesulphonic, acids.

Specific examples of A3RAg which may be employed according to general formula (IV) of the present invention include, without being limited thereto, N⁶-2-(4-aminophenyl)ethyladenosine (APNEA), N⁶-(4-amino-3-iodobenzyl) adenosine-5′-(N-methyluronamide) (AB-MECA) and N⁶-(2-iodobenzyl)-adenosine-5′-N-methlyuronamide (IB-MECA) and preferably 2-chloro-N⁶-(2-iodobenzyl)-adenosine-5′-N-methlyuronamide (Cl-IB-MECA).

According to another embodiment, the A3RAg may be an oxide derivative of adenosine, such as N⁶-benzyladenosine-5′-N-alkyluronamide-N¹-oxide or N⁶-benzyladenosine-5′-N-dialkyluronamide-N¹-oxide, wherein the 2-purine position may be substituted with a alkoxy, amino, alkenyl, alkynyl or halogen.

The present invention also provides a method for a therapeutic treatment comprising administering to an individual in need, one or more A3RAg in an amount effective for achieving a therapeutic effect, the therapeutic effect comprises activation of NK cells in said individual.

Further, provided by the present invention is a method for treatment of a disease comprising administering to an individual in need of such treatment NK cells a priori activated with an effective amount of A3RAg. In accordance with one embodiment, the NK cells are autologous cells a priori withdrawn from the same individual and then activated ex vivo by contacting them with an amount of an A3RAg effective to activate them, and then reintroduced to the individual, by a suitable parenteral administration. Alternatively, the NK cells may at times be obtained from a donor individual either after activation in vivo by administering the A3RAg to the donor individual a sufficient time prior to withdrawal of the cells, or activating the cells ex vivo as above, or both. Methods for withdrawal of relatively purified NK cells populations from an individual and their ex vivo culture are known in the art and need not be further elaborated herein.

The A3RAg may be formulated in different ways. It may be formulated as such or converted into a pharmaceutically acceptable salt. It can be administered alone or in combination with pharmaceutically acceptable carriers, diluents, excipients, additives and adjuvants (generally referred to herein as pharmaceutically acceptable additives, defined hereinafter).

When providing A3RAg to an individual for in vivo treatment or to an animal model for ex vivo treatment, it is preferably formulated for oral delivery. However, other methods, of administration are also suitable such as parenteral administration including intravenous, subcutaneous, intramuscular intramedullary injection, intraarterial, intraperitoneally and intranasal administration as well as intrathecal and by infusion techniques. For oral administration, A3RAg with good oral bioavailability may preferably be chosen. Screening for an A3Rag with good oral bioavailability and good effectivity in achieving the desired therapeutic effect, is a routine task within easy reach of the artisan.

When administering A3RAg orally, it is preferably formulated for administration as a tablet, a suspension, a solution, an emulsion, a capsule, a powder, a syrup and the like.

When administering A3RAg parenterally, it will generally be formulated in a unit dosage injectable form (solution, suspension, emulsion) and will include sterile aqueous solutions or dispersions and sterile powders for reconstitution into sterile injectable solutions or dispersions.

It is noted that humans are treated generally longer than experimental animals as exemplified herein, which treatment has a length proportional to the length of the disease process. The doses may be single doses or multiple doses over a period of time, e.g. several days and may depend of physical characteristics such as the high, weight, gender of the individual to be treated. Generally, the administrated doses are preferably unit dosage form. The treatment generally has a length which may be contingent on the length and stage of the disease process and active agent effectiveness and the patient species being treated.

Thus, the present invention also provides pharmaceutical compositions comprising one or more A3RAg in an amount effective to achieve a therapeutic effect, the therapeutic effect comprising activation of NK cells and optionally pharmaceutically acceptable additives.

By the term “pharmaceutically acceptable additives” it is meant any inert, non-toxic materials, which do not react with A3RAg and which are typically added to formulations as diluents or carriers or to give form or consistency to the formulation to give it a specific form, e.g. in pill form, as a simple syrup, aromatic powder, and other various forms. The additives may also be substances for providing the formulation with stability (e.g. preservatives) or for providing the formulation with an edible flavor etc.

The additives can be any of those conventionally used and is limited only by chemico-physical considerations, such as solubility and lack of reactivity with A3RAg, and by the route of administration. The choice of additive will be determined in part by the specific A3RAg employed, as well as by the particular method used to administer the composition. Accordingly, the additives may include excipients, colorants, diluents, buffering agents, disintegrating agents, moistening agents, preservatives, flavoring agents, and pharmacologically compatible carriers. In addition, the additive may be an adjuvant, which, by definition are substances affecting the action of the active ingredient in a predictable way.

Accordingly, pharmaceutical compositions suitable for oral administration may consist of (a) liquid solutions, where an effective amount of A3RAg dissolved in diluents, such as water, saline, natural juice, alcohols, syrups, etc.; (b) capsules (e.g. the ordinary hard- or soft-shelled gelatin type containing, for example, surfactants, lubricants, and inert fillers), tablets, lozenges (wherein A3RAg is in a flavor, such as sucrose and acacia or tragacanth or the A3RAg is in an inert base, such as gelatin and glycerin), and troches, each containing a predetermined amount of A3RAg as solids or granules; (c) powders; (d) suspensions in an appropriate liquid; (e) suitable emulsions; (f) liposome formulation; and others.

In addition, A3RAg may also be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Pharmaceutical compositions formulated for parenteral administration may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Oils such as petroleum, animal, vegetable, or synthetic oils and soaps such as fatty alkali metal, ammonium, and triethanolamine salts, and suitable detergents may also be used for parenteral administration. Further, in order to minimize or eliminate irritation at the site of injection, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The invention will now be exemplified in the following. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described hereinbelow.

### SPECIFIC EXAMPLES

The effect of the A3 adenosine receptor agonist 2-chloro-N⁶-(2-iodobenzyl)-adenosine-5′-N-methly-uronamide (Cl-IB-MECA)., on the in vitro and in vivo activity of NK cells, was tested.

In vitro Studies

In this set of experiments, the effect of the synthetic adenosine A3 receptor agonist (A3ARAg), 2-chloro-N⁶-(3-iodobenzyl)-adenosine-5′-N-methyl-uronamide (Cl-IB-MECA) on the activity of murine spleenocytes or human peripheral blood mononuclear cells was tested.

The A3 adenosine receptor antagonist 9-chloro-2-(2-furanyl)-5-[(phenylacetyl)amino] [1,2,4,] -triazolo[1,5-c] quinazoline (MRS-1220) was used to prove the specific binding of Cl-IB-MECA to the A3AR.

All drugs were purchased from RBI Massachusetts, USA. A stock solution was prepared by dissolving 5 mg Cl-IB-MECA in 1 ml DMSO. Farther dilutions were performed in RPMI.

Murine splenocytes were derived from spleens of ICR mice and human mononuclear cells were separated by Ficoll-Hypaque gradient from heparinized blood of healthy normal volunteers.

The effect of Cl-IB-MECA on the activity of human peripheral blood NK cells was assayed by a standard 4 h ⁵¹Cr-release assay using K562 leukemia cells as targets. Splenocytes or human mononuclear cells were cultured at a concentration of 5×10⁵ cells/well in 96 well round bottom plates, and used as the effector (E) cells. The cells were preincubated with 10 nM Cl-IB-MECA for 18 hours, then the agonist was washed out from the wells and the cells were re-suspended in RPMI containing 5% FCS. K562 cells were used as the targets (T) and were labeled with 100 μci of Na₂[⁵¹Cr]O₄ at 37° C., for 1 hr.

After extensive washing to remove the excess ⁵¹Cr, target cells (1×10⁴) were re-suspended in RPMI and mixed with the effector cells at an E:T ratio of 1:50 in a total volume of 200 μl (triplicate assays). After 4 hours of incubation at 37° C. in 5% CO₂, plates were centrifuged, and the supernatants were counted in a gamma counter (LKB).

NK cytotoxicity was calculated using the following equation (CPM: counts per minute):

$$\% Lysis = \frac{CPM sample - CPM spontaneous}{CPM maximal - CPM spontaneous} \times 100$$

CPM sample, CPM spontaneous and CPM maximal were determined by measuring the CPM of the supernatants of the target cells in the presence of Na₂[⁵¹Cr]O₄, the assay medium or in the presence of 1% triton, respectively. It should be noted that spontaneous release was below 10% of the maximal release throughout this experiment.

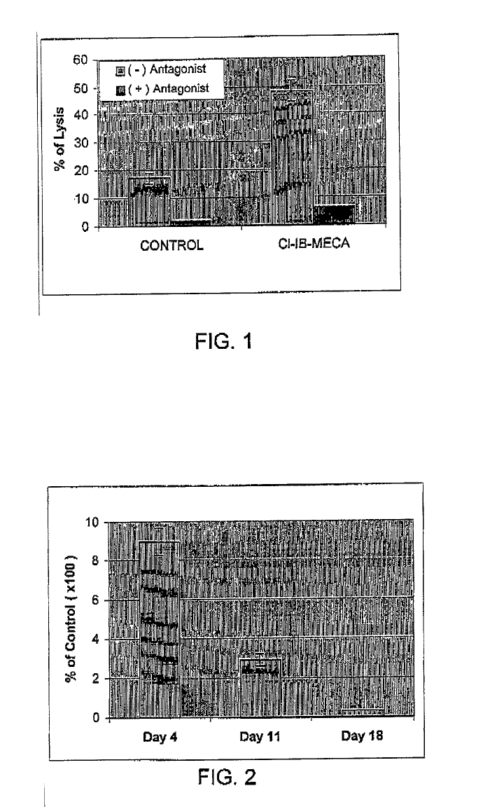

A significant dose dependent increase in the activity of natural killer cells following preincubation with CI-IB-MECA was observed (FIG. 1). Introduction of the A3 adenosine receptor antagonist MRS-1220 abolished the stimulatory effect, exhibiting the specific activation of the A3 adenosine receptor by Cl-IB-MECA.

In vivo Studies

ICR mice were orally administered for two consecutive days with 6 μg/kg body weight of Cl-IB-MECA. After 4, 11 and 18 days, mice were sacrificed and spleens were removed. Splenocytes were separated and tested for NK activity using the ⁵¹Cr assay as described above.

As shown in FIG. 2, It was found that Cl-IB-MECA induces potentiation of NK cell activity following oral administration to the mice. In particular, after 4 days a marked increase in the NK activity of splenocytes derived from treated mice was observed. After 11 days, a high activity was still observed, while following 18 days only slight increase is shown.

In a further study C57B1/6J mice were used as the model mice which will develop metastatic lung foci after 15 days. The C57B1/6J mice were inoculated intravenously with B-16 melanoma cells (2.5×10⁵) and treated daily orally with 6 or 9 μg/kg body weight of Cl-IB-MECA (starting one day after tumor inoculation). After 15 days, the mice were sacrificed and spleens were removed.

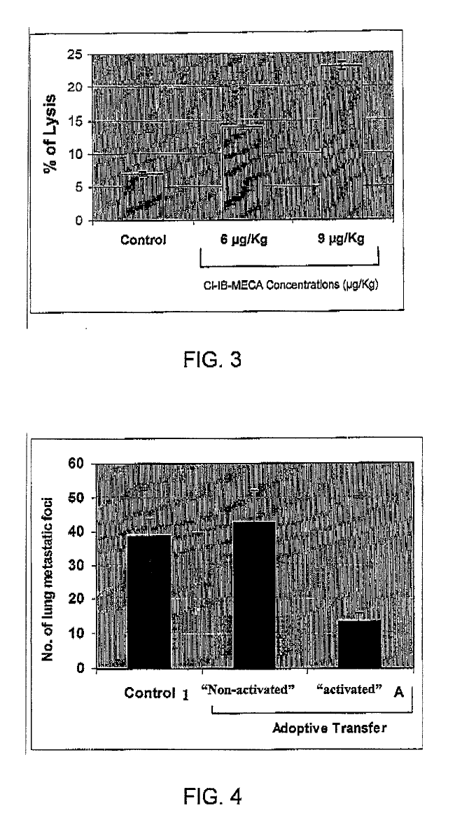

Splenocytes were separated and tested for NK activity using the ⁵¹Cr assay as described above. A marked increase in the NK activity of splenocytes derived from 6 and 9 μg/kg Cl-IB-MECA treated mice was observed in comparison to control group which as treated with the vehicle only (FIG. 3). The splenocytes derived from the group treated with 6 μg/kg body weight of Cl-IB-MECA were designated as “activated” cells and those derived from the control group were designated as “non-activated” splenocytes.

In another experiment, the capability of the “activated” splenocytes to act in vivo against melanoma cells was examined. The “non-activated” and “activated” splenocytes were engrafted to mice that were inoculated 4 days earlier with B-16 melanoma cells (2.5×10⁵). As the control served mice that were inoculated with the B-16 melanoma cells however not engrafted with splenocytes.

The mice were sacrificed on day 15 and their lungs were removed. The number of lung melanoma foci was counted, the results for which are depicted in FIG. 4.

As may be viewed from FIG. 4, the mice engrafted with “activated” splenocytes showed a marked inhibition in development of lung metastatic foci as compared to the number of foci developed with mice engrafted with “non-activated” splenocytes or with the control group. These results prove that Cl-IB-MECA activate NK cells.

1. A method for activating natural killer (NK) cells in an individual comprising administering said individual with an effective amount of one or more adenosine A3 receptor agonists (A3RAg).

2. The method of claim 1, wherein said A3RAg is a compound of the general formula (I): 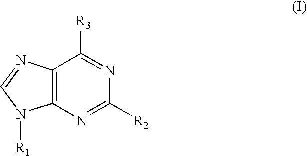
wherein,
R₁ represents an alkyl, hydroxyalkyl, carboxyalkyl or cyanoalkyl or a group of the following general formula (II): 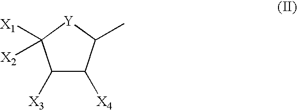
in which:
Y represents an oxygen, sulfur of carbon atom;
X₁ represents H, alkyl, R^{a}R^{b}NC(═O)— or HOR^{c}—, wherein
R^{a} and R^{b} may be the same or different and are selected from the group consisting of hydrogen, alkyl, amino, haloalkyl, aminoalkyl, BOC-aminoalkyl, and cycloalkyl or are joined together to form a heterocyclic ring containing two to five carbon atoms; and
R^{c} is selected from the group consisting of alkyl, amino, haloalkyl, aminoalkyl, BOC-aminoalkyl, and cycloalkyl;
X₂ is H, hydroxyl, alkylamino, alkylamido or hydroxyalkyl;
X₃ and X₄ represent independently hydrogen, hydroxyl, amino, amido, azido, halo, alkyl, alkoxy, carboxy, nitrilo, nitro, trifluoro, aryl, alkaryl, thio, thioester, thioether, —OCOPh, —OC(═S)OPh or both X₃ and X₄ are oxygens connected to >C═S to form a 5-membered ring, or X₂ and X₃ form the ring of formula (III): 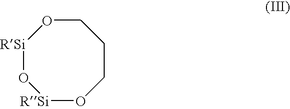
where R′ and R″ represent independently an alkyl group;
R₂ is selected from the group consisting of hydrogen, halo, alkylether, amino, hydrazido, alkylamino, alkoxy, thioalkoxy, pyridylthio, alkenyl; alkynyl, thio, and alkylthio; and
R₃ is a group of the formula —NR₄R₅ wherein
R₄ is a hydrogen atom or a group selected from alkyl, substituted alkyl or aryl-NH—C(Z)—, with Z being O, S, or NR^{a} with R^{a} having the above meanings; wherein when R₄ is hydrogen than
R₅ is selected from the group consisting of R- and S-1-phenylethyl, benzyl, phenylethyl or anilide groups unsubstituted or substituted in one or more positions with a substituent selected from tie group consisting of alkyl, amino, halo, haloalkyl, nitro, hydroxyl, acetoamido, alkoxy, and sulfonic acid or a salt thereof; benzodioxanemethyl, fururyl, L-propylalanyl-aminobenzyl, β-alanylamino-benzyl, T-BOC-β-alanylaminobenzyl, phenylamino, carbamoyl, phenoxy or cycloalkyl; or R₅ is a group of the following formula: 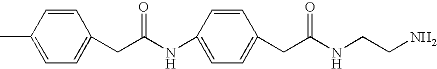
or when R₄ is an alkyl or aryl-NH—C(Z)—, then, R₅ is selected from the group consisting of heteroaryl-NR^{a}—C(Z)—, heteroaryl-C(Z)—, alkaryl-NR^{a}—C(Z)—, alkaryl-C(Z)—, aryl-NR—C(Z)— and aryl-C(Z)—; Z representing an oxygen, sulfor or amine;
or a pharmaceutically acceptable salt of the above compound.

3. The method of claim 2, wherein said A3RAg is a nucleoside derivative of the general formula (IV): 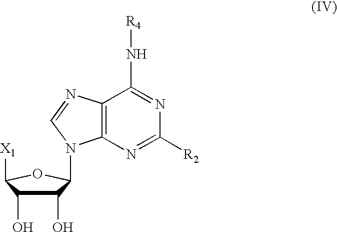
wherein X₁, R₂ and R₄ are as defined in claim 2.

4. The method of claim 3, wherein A3RAg is selected from the group consisting group consisting of N⁶-2-(4-aminophenyl)ethyladenosine (APNEA), N⁶-(4-amino-3-iodobenzyl) adenosine-5′-(N-methyluronamide) (AB-MECA) and N⁶-(2-iodobenzyl)-adenosine-5′-N-methly-uronamide (IB-MECA) and 2-chloro-N⁶-(2-iodobenzyl)-adenosine-5′-N-methly-uronamide (Cl-IB-MECA).

5. The method of claim 4, wherein A3RAg is IB-MECA or Cl-IB-MECA.

6. The method of claim 1, wherein said A3RAg is N⁶-benzyladenosine-5′-N-alkyluronamide-N¹-oxide or N⁶-benzyladenosine-5′-N-dialkyluronamide-N¹-oxide, both optionally substituted at the 2-purine position with an alkoxy, amino, alkenyl, alkynyl or halogenoxide group.

7. The method of claim 1 wherein said A3RAg is administered orally to said individual.

8. The method of claim 1, wherein said A3RAg is injected to said individual.

9. A method for a therapeutic treatment comprising administering to an individual in need, one or more A3RAg in an amount effective for achieving a therapeutic effect, the therapeutic effect comprises activation of NK cells in said individual.

10. The method of claim 9, wherein said A3RAg is a compound of the general formula (I): 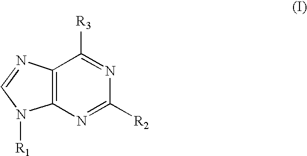
wherein,
R₁ represents an alkyl, hydroxyalkyl, carboxyalkyl or cyanoalkyl or a group of the following general formula (II): 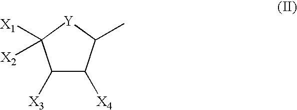
in which:
Y represents an oxygen, sulfur of carbon atom;
X₁ represents H, alkyl, R^{a}R^{b}NC(═O)— or HOR^{c}—, wherein
R^{a} and R^{b} may be the same or different and are selected from the group consisting of hydrogen, alkyl, amino, haloalkyl, aminoalkyl, BOC-aminoalkyl, and cycloalkyl or are joined together to form a heterocyclic ring containing two to five carbon atoms; and
R^{c} is selected from the group consisting of alkyl, amino, haloalkyl, aminoalkyl, BOC-aminoalkyl, and cycloalkyl;
X₂ is H, hydroxyl, alkylamino, alkylamido or hydroxyalkyl;
X₃ and X₄ represent independently hydrogen, hydroxyl, amino, amido, azido, halo, alkyl, alkoxy, carboxy, nitrilo, nitro, trifluoro, aryl, alkaryl, thio, thioester, thioether, —OCOPh, —OC(═S)OPh or both X₃ and X₄ are oxygens connected to >C═S to form a 5-membered ring, or X₂ and X₃ form the ring of formula (III): 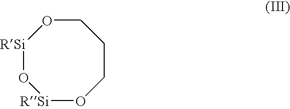
where R′ and R″ represent independently an alkyl group;
R₂ is selected from the group consisting of hydrogen, halo, alkylether, amino, hydrazido, alkylamino, alkoxy, thioalkoxy, pyridylthio, alkenyl; alkynyl, thio, and alkylthio; and
R₃ is a group of the formula —NR₄R₅ wherein
R₄ is a hydrogen atom or a group selected from alkyl, substituted alkyl or aryl-NH—C(Z)—, with Z being O, S, or NR^{a} with R^{a} having the above meanings; wherein when R₄ is hydrogen than
R₅ is selected from the group consisting of R- and S-1-phenylethyl, benzyl, phenylethyl or anilide groups unsubstituted or substituted in one or more positions with a substituent selected from the group consisting of alkyl, amino, halo, haloalkyl, nitro, hydroxyl, acetoamido, alkoxy, and sulfonic acid or a salt thereof; benzodioxanemethyl, fururyl, L-propylalanyl-aminobenzyl, β-alanylamino-benzyl, T-BOC-β-alanylaminobenzyl, phenylamino, carbamoyl, phenoxy or cycloalkyl; or R₅ is a group of the following formula: 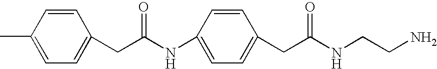
or when R₄ is an alkyl or aryl-NH—C(Z)—, then, R₅ is selected from the group consisting of heteroaryl-NR^{a}—C(Z)—, heteroaryl-C(Z)—, alkaryl-NR^{a}—C(Z)—, alkaryl-C(Z)—, aryl-NR—C(Z)— and aryl-C(Z)—; Z representing an oxygen, sulfor or amine;
or a pharmaceutically acceptable salt of the above compound.

11. The method of claim 10, wherein said A3RAg is a nucleoside derivative of the general formula (IV): 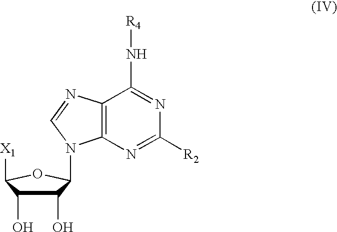
wherein X₁, R₂ and R₄ are as defined.

12. The method of claim 11, wherein said A3RAg is selected from the group consisting group consisting of N⁶-2-(4-aminophenyl)ethyladenosine (APNEA), N⁶-(4-amino-3-iodobenzyl) adenosine-5′-(N-methyluronamide) (AB-MECA) and N⁶-(2-iodobenzyl)-adenosine-5′-N-methlyuronamide (IB-MECA) and 2-chloro-N⁶-(2-iodobenzyl)-adenosine-5′-N-methlyuronamide (Cl-IB-MECA).

13. The method of claim 12, wherein said A3RAg is Cl-IB-MECA.

14. The method of claim 9, wherein said A3RAg is N⁶-benzyladenosine-5′-N-alkyluronamide-N¹-oxide or N⁶-benzyladenosine-5′-N-dialkyluronamide-N¹-oxide, both optionally substituted at the 2-purine position with an alkoxy, amino, alkenyl, alkynyl or halogenoxide group.

15. The method of claim 9, wherein said A3RAg is orally administered to said individual.

16. The method of claim 9, wherein said A3RAg is injected to said individual.

17. A method for treatment of a disease comprising administering to an individual in need of such treatment NK cells a priori activated with an effective amount of at least one A3RAg.

18. The method according to claim 17, wherein said NK cells are autologous cells, the method comprising withdrawing NK cells from the individual, contacting said cells with an amount of an A3RAg effective to activate said NK cells and injecting the activated NK cells to the individual.

19. The method of claim 17, wherein said A3RAg is a compound of the general formula (I): 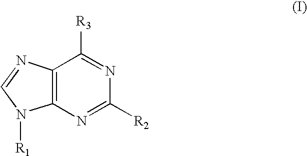
wherein,
R₁ represents an alkyl, hydroxyalkyl, carboxyalkyl or cyanoalkyl or a group of the following general formula (II): 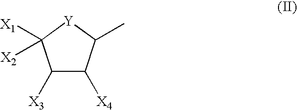
in which:
Y represents an oxygen, sulfur of carbon atom;
X₁ represents H, alkyl, R^{a}R^{b}NC(═O)— or HOR^{c}—, wherein
R² and R^{b} may be the same or different and are selected from the group consisting of hydrogen alkyl, amino, haloalkyl, aminoalkyl, BOC-aminoalkyl, and cycloalkyl or are joined together to form a heterocyclic ring containing two to five carbon atoms; and
R^{c} is selected from the group consisting of alkyl, amino, haloalkyl, aminoalkyl, BOC-aminoalkyl, and cycloalkyl;
X₂ is H, hydroxyl, alkylamino, alkylamido or hydroxyalkyl;
X₃ and X₄ represent independently hydrogen, hydroxyl, amino, amido, azido, halo, alkyl, alkoxy, carboxy, nitrilo, nitro, trifluoro, aryl, alkaryl, thio, thioester, thioether, —OCOPh, —OC(═S)OPh or both X₃ and X₄ are oxygens connected to >C═S to form a 5-membered ring, or X₂ and X₃ form the ring of formula (III): 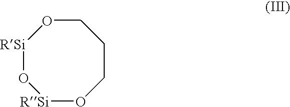
where R′ and R″ represent independently an alkyl group;
R₂ is selected from the group consisting of hydrogen, halo, alkylether, amino, hydrazido, alkylamino, alkoxy, thioalkoxy, pyridylthio, alkenyl; alkynyl, thio, and alkylthio; and
R₃ is a group of the formula —NR₄R₅ wherein
R₄ is a hydrogen atom or a group selected from alkyl, substituted alkyl or aryl-NH—C(Z)—, with Z being O, S, or NR^{a} with R^{a} having the above meanings; wherein when R₄ is hydrogen than
R₅ is selected from the group consisting of R- and S-1-phenylethyl, benzyl, phenylethyl or anilide groups unsubstituted or substituted in one or more positions with a substituent selected from the group consisting of alkyl, amino, halo, haloalkyl, nitro, hydroxyl, acetoamido, alkoxy, and sulfonic acid or a salt thereof; benzodioxanemethyl, fururyl, L-propylalanyl-aminobenzyl, β-alanylamino-benzyl, T-BOC-β-alanylaminobenzyl, phenylamino, carbamoyl, phenoxy or cycloalkyl; or R₅ is a group of the following formula: 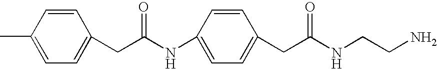
or when R₄ is an alkyl or aryl-NH—C(Z)—, then, R₅ is selected from the group consisting of heteroaryl-NR^{a}—C(Z)—, heteroaryl-C(Z)—, alkaryl-NR^{a}—C(Z)—, alkaryl-C(Z)—, aryl-NR—C(Z)— and aryl-C(Z)—; Z representing an oxygen, sulfor or amine;
or a pharmaceutically acceptable salt of the above compound.

20. The method of claim 19, wherein said A3RAg is a nucleoside derivative of the general formula (IV): 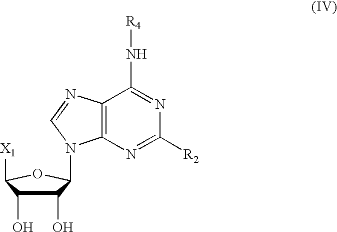
wherein X₁, R₂ and R₄ are as defined.

21. The method of claim 20, wherein said A3RAg is selected from the group consisting group consisting of N⁶-2-(4-aminophenyl)ethyladenosine (APNEA), N⁶-(4-amino-3-iodobenzyl) adenosine-5′-(N-methyluronamide) (AB-MECA) and N⁶-(2-iodobenzyl)-adenosine-5′-N-methlyuronamide (IB-MECA) and 2-chloro-N⁶-(2-iodobenzyl)-adenosine-5′-N-methlyuronamide (Cl-IB-MECA).

22. The method of claim 21, wherein said A3RAg is Cl-IB-MECA.

23. The method of claim 17, wherein said A3RAg is N⁶-benzyladenosine-5′-N-alkyluronamide-N¹-oxide or N⁶-benzyladenosine-5′-N-dialkyluronamide-N¹-oxide, both optionally substituted at the 2-purine position with an alkoxy, amino, alkenyl, alkynyl or halogenoxide group.

24. The method of claim 17, comprising administering an amount of an A3RAg to a donor individual effective to activate the NK cells in the donor individual, withdrawing the activated NK cells from the donor individual and administering the activated NK cells to a recipient individual.

25. The method of claim 24, wherein said A3RAg is orally administered to said donor individual.

26. The method of claim 24, wherein said A3RAg is administered to said donor individual by injection.

27. A pharmaceutical composition comprising one or more A3RAg in an amount effective to achieve a therapeutic effect, the therapeutic effect comprising activation of NK cells, the pharmaceutical composition optionally comprising physiologically acceptable additives.

28. The pharmaceutical composition of claim 27, wherein said A3RAg is a compound of the general formula (I): 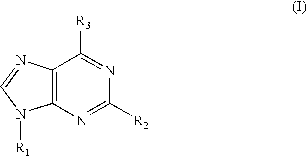
wherein,
R₁ represents an alkyl, hydroxyalkyl, carboxyalkyl or cyanoalkyl or a group of the following general formula (II): 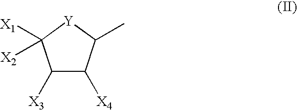
in which:
Y represents an oxygen, sulfur of carbon atom;
X₁ represents H, alkyl, R^{a}R^{b}NC(═O)— or HOR^{c}—, wherein
R^{a} and R^{b} may be the same or different and are selected from the group consisting of hydrogen, alkyl, amino, haloalkyl, aminoalkyl, BOC-aminoalkyl, and cycloalkyl or are joined together to form a heterocyclic ring containing two to five carbon atoms; and
R^{c} is selected from the group consisting of alkyl, amino, haloalkyl, aminoalkyl, BOC-aminoalkyl, and cycloalkyl;
X₂ is H, hydroxyl, alkylamino, alkylamido or hydroxyalkyl;
X₃ and X₄ represent independently hydrogen, hydroxyl, amino, amido, azido, halo, alkyl, alkoxy, carboxy, nitrilo, nitro, trifluoro, aryl, alkaryl, thio, thioester, thioether, —OCOPh, —OC(═S)OPh or both X₃ and X₄ are oxygens connected to >C═S to form a 5-membered ring, or X₂ and X₃ form the ring of formula (III): 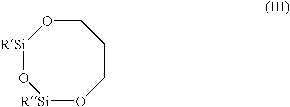
where R′ and R″ represent independently an alkyl group;
R₂ is selected from the group consisting of hydrogen, halo, alkylether, amino, hydrazido, alkylamino, alkoxy, thioalkoxy, pyridylthio, alkenyl; alkynyl, thio, and alkylthio; and
R₃ is a group of the formula —NR₄R₅ wherein
R₄ is a hydrogen atom or a group selected from alkyl, substituted alkyl or aryl-NH—C(Z)—, with Z being O, S, or NR^{a} with R^{a} having the above meanings; wherein when R₄ is hydrogen than
R₅ is selected from the group consisting of R- and S-1-phenylethyl, benzyl, phenylethyl or anilide groups unsubstituted or substituted in one or more positions with a substituent selected from the group consisting of alkyl, amino, halo, haloalkyl, nitro, hydroxyl, acetoamido, alkoxy, and sulfonic acid or a salt thereof; benzodioxanemethyl, fururyl, L-propylalanyl-aminobenzyl, β-alanylamino-benzyl, T-BOC-β-alanylaminobenzyl, phenylamino, carbamoyl, phenoxy or cycloalkyl; or R₅ is a group of the following formula: 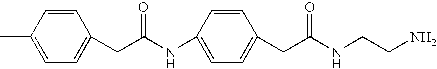
or when R₄ is an alkyl or aryl-NH—C(Z)—, then, R₅ is selected from the group consisting of heteroaryl-NR^{a}—C(Z)—, heteroaryl-C(Z)—, alkaryl-NR^{a}—C(Z)—, alkaryl-C(Z)—, aryl-NR—C(Z)— and aryl-C(Z)—; Z representing an oxygen, sulfor or amine;
or a pharmaceutically acceptable salt of the above compound.

29. The pharmaceutical composition of claim 28, wherein said A3RAg is a nucleoside derivative of the general formula (IV): 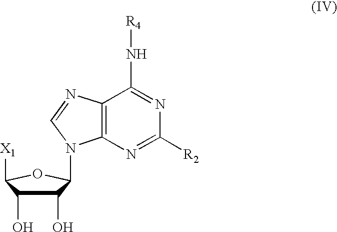
wherein X₁, R₂ and R₄ are as defined in claim 2.

30. The pharmaceutical composition of claim 29, wherein A3RAg is selected from the group consisting group consisting of N⁶-2-(4-aminophenyl)ethyladenosine (APNEA), N⁶-(4-amino-3-iodobenzyl) adenosine-5′-(N-methyluronamide) (AB-MECA) and N⁶-(2-iodobenzyl)-adenosine-5′-N-methly-uronamide (IB-MECA) and 2-chloro-N⁶-(2-iodobenzyl)-adenosine-5′-N-methly-uronamide (Cl-IB-MECA).

31. The pharmaceutical composition of claim 30, wherein A3RAg is Cl-IB-MECA.

32. The pharmaceutical composition of claim 27, wherein said A3RAg is N⁶-benzyladenosine-5′-N-alkyluronamide-N¹-oxide or N⁶-benzyladenosine-5′-N-dialkyluronamide-N¹-oxide, both optionally substituted at the 2-purine position with an alkoxy, amino, alkenyl, alkynyl or halogenoxide group.

33. The pharmaceutical composition of claim 27, wherein said A3RAg is formulated for oral administration to said individual.

34. The pharmaceutical composition of claim 27, wherein said A3RAg is formulated for injection to said individual.

35. The pharmaceutical composition of claim 27, in a single dosage unit form.
